(19) United States
(12) Patent Application Publication
(10) Pub. No.: US 2024/0238508 A1
(43) Pub. Date: Jul. 18, 2024

(54) Screen Control Method for Syringe Pump, Apparatus, Device and Storage Medium

(71) Applicant: Medcaptain Medical Technology Co., Ltd., Shenzhen (CN)

(72) Inventors: Longyu WU, Shenzhen (CN); Ping LI, Shenzhen (CN)

(73) Assignee: Medcaptain Medical Technology Co., Ltd., Shenzhen (CN)

(21) Appl. No.: 18/410,108

(22) Filed: Jan. 11, 2024

(30) Foreign Application Priority Data
Jan. 13, 2023 (CN) 202310077640.1

(51) Int. Cl. A61M 5/142

(52) U.S. Cl. CPC A61M 5/142; A61M 2205/33; A61M 2205/3327; A61M 2205/502

(57) ABSTRACT

A screen control method is provided for a syringe pump, along with a corresponding apparatus, device and storage medium. The syringe pump includes a pump door and a pump body, and the pump door is provided with a display screen. The pump body is configured to receive a syringe, and the pump door is slidably connected to the pump body. The method is applied to the pump door and includes determining, according to a sliding operation performed by a user on the pump door, a screen control instruction corresponding to the sliding operation, where the sliding operation is configured to indicate sliding of the pump door; and controlling the display screen according to the screen control instruction.

### REFERENCE TO RELATED APPLICATIONS

The invention claims priority of Chinese patent application Ser. No. 202310077640.1, entitled “SYRINGE PUMP BASED SCREEN CONTROL METHOD, APPARATUS, DEVICE AND STORAGE MEDIUM” filed with the China National Intellectual Property Administration on Jan. 13, 2023, which is incorporated herein by reference in its entirety.

### TECHNICAL FIELD

This invention generally relates to the technical field of automatic control, and more particularly, to a screen control method for a syringe pump, apparatus, device and storage medium.

### BACKGROUND ART

In infusion scenarios, Syringe pumps equipped with injectors are widely used. With the increasing use of syringe pumps, there are growing demands on their functionalities.

Currently, syringe pumps are fitted with a display screen, which may be set on the pump door, and users can operate the display screen to view the injection process of the syringe. Therefore, how to efficiently control the screen in the process of using the syringe pump has become an urgent problem to be solved at present.

### SUMMARY OF THE INVENTION

The invention provides a screen control method for a syringe pump, apparatus, device and storage medium, which can improve the efficiency of controlling the screen.

In a first aspect, the invention provides a screen control method for a syringe pump, applied to a pump door in the syringe pump, the syringe pump comprising the pump door and a pump body, the pump door being fitted with a display screen, the pump body being configured to place a syringe, and the pump door being slidably connected to the pump body; the method comprising:
determining, according to a sliding operation performed by a user on the pump door, a screen control instruction corresponding to the sliding operation; wherein the sliding operation indicates sliding of the pump door; and
controlling the display screen according to the screen control instruction.

In a second aspect, the invention provides a screen control apparatus for a syringe pump, applied to a pump door in the syringe pump, the syringe pump comprising the pump door and a pump body, the pump door being fitted with a display screen, the pump body being configured to place a syringe, and the pump door being slidably connected to the pump body; the device comprising:
an instruction determination module configured to determine, according to a sliding operation performed by a user on the pump door, a screen control instruction corresponding to the sliding operation; wherein the sliding operation indicates sliding of the pump door; and
a screen control module configured to control the display screen according to the screen control instruction.

In a third aspect, the invention provides an electronic device, comprising a processor, and a memory in communication with the processor; wherein
the memory is configured to store computer-executable instructions; and
the processor is configured to execute the computer-executable instructions stored in the memory, to realize a screen control method for a syringe pump, comprising:
determining, according to a sliding operation performed by a user on a pump door of the syringe pump, a screen control instruction corresponding to the sliding operation; wherein the sliding operation indicates sliding of the pump door; and
controlling the display screen according to the screen control instruction.

In a fourth aspect, the invention provides a computer-readable storage medium, wherein the computer-readable storage medium stores computer-executable instructions, and the computer-executable instructions are executed by the processor to realize the screen control method for the syringe pump as described in the first aspect of the invention.

In a fifth aspect, the invention provides a syringe pump, which is configured to implement the screen control method as described in the first aspect of the invention.

The invention provides a screen control method for a syringe pump, device, apparatus and storage medium. The syringe pump includes a pump door and a pump body, and the pump door is fitted with a display screen to display information relating to, for example, the injection process and the injected drug. The pump body is configured to place a syringe, and the pump door is slidably connected to the pump body, and the pump door slides to expose the syringe in the pump body. The user can perform a sliding operation on the pump door, and different sliding operations can correspond to different screen control instructions, so that automatic control of the display screen can be realized according to the screen control instructions, thereby solving the problem of low control efficiency caused by the existing technology in which the user needs to manually make clicks and other operations on the screen for control. Besides, by sliding the pump door to control the screen, it can satisfy the user's need to observe the information on the screen and the information on the syringe at the same time, thereby improving the control efficiency of the screen and enhancing the user's experience.

### BRIEF DESCRIPTION OF THE DRAWINGS

The accompanying drawings herein, which are incorporated into and form a part of the specification, illustrate embodiments consistent with the invention and are used in conjunction with the specification to explain the principles of the invention.

FIG. 1 shows a schematic diagram of a syringe pump with a clamshell pump door according to an embodiment of the invention.

FIG. 2 shows a flowchart of a screen control method for a syringe pump according to an embodiment of the invention.

FIG. 3 shows a schematic diagram of a syringe pump with a sliding pump door according to an embodiment of the invention.

FIG. 4 shows a flowchart of a screen control method for the syringe pump according to an embodiment of the invention.

FIG. 5 shows a schematic diagram of a syringe pump with a sliding pump door according to an embodiment of the invention.

FIG. 6 shows a block diagram of a screen control apparatus for the syringe pump according to an embodiment of the invention.

FIG. 7 provides a block diagram of a screen control apparatus for the syringe pump according to an embodiment of the invention.

FIG. 8 provides a block diagram of an electronic device according to an embodiment of the invention.

FIG. 9 provides a block diagram of an electronic device according to an embodiment of the invention.

By means of the above accompanying drawings, definite embodiments of the invention have been shown, and will be described in greater detail later. These accompanying drawings and textual descriptions are not intended to limit the scope of the invention idea in any way, but rather to illustrate the concepts of the invention for those skilled in the art by reference to particular embodiments.

### DETAILED DESCRIPTION

In order to make the purpose, technical solutions and advantages of the invention clearer, the following embodiments of the invention will be described in further detail in conjunction with the accompanying drawings.

It should be clear that the described embodiments are only some of the embodiments of the invention, and not all of the embodiments. According to the embodiments in the invention, all other embodiments obtained by a person of ordinary skill in the art without creative labor fall within the protection scope of the invention.

When the following description relates to the accompanying drawings, the same numerals in different accompanying drawings indicate the same or similar elements unless otherwise indicated. The embodiments described in the following example embodiments do not represent all embodiments consistent with the invention. Rather, they are only examples of apparatuses and methods that are consistent with some aspects of the invention as detailed in the appended claims.

In the description of the invention, it is to be understood that the terms “first,” “second,” “third,” and the like are used only to distinguish similar objects and need not be used to describe a particular order or order, nor are they to be understood as indicating or implying relative importance. For those of ordinary skill in the art, the specific meanings of the above terms in the invention may be understood according to the specific circumstances. Furthermore, in the description of the invention, unless otherwise indicated, “plurality” means two or more. “And/or”, which describes the correlation relationship of associated objects, indicates that three relationships can exist. For example, A and/or B can indicate: A alone, both A and B, and B alone. The character “/” generally indicates that the associated objects are in an “or” relationship.

It should be noted that, due to the limitation of space, the invention specification does not exhaust all the optional embodiments, and the person skilled in the art should be able to think of, after reading the invention specification, that as long as the technical features do not contradict each other, then any combination of the technical features can constitute an optional embodiment. Each embodiment is described in detail below.

In a clinical infusion scenario, drugs need to be injected using equipment such as syringe pumps, where syringes are placed and labeled with information such as drug labels. A healthcare worker needs to frequently check the drug labels on the syringe to understand the injection of the drug.

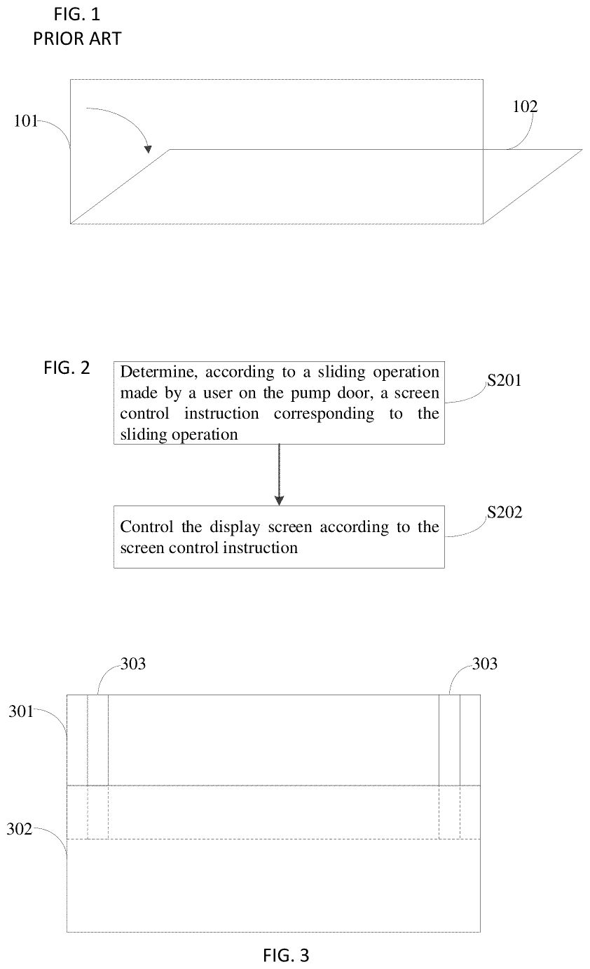

The pump door on the syringe pump can be arranged with a display screen, so healthcare workers can operate the display screen to view the progress of drug injection and patient personal information. At present, the syringe pump arranged with a display screen is a syringe pump with a clamshell pump door, however, the conventional clamshell pump door covers the syringe. FIG. 1 shows a schematic diagram of a syringe pump with a clamshell pump door. In FIG. 1, a pump body 101 and a pump door 102 are connected axially at a bottom edge of the pump body. The pump door 102 is disposed in an initial position when it is not being used, and the initial position of the pump door 102 just shields the pump body 101. A syringe may be disposed in the pump body, and when the pump door 102 is disposed in the initial position, the syringe is obscured. The pump door 102 may be downturned along a bottom edge of the pump body 101, and the syringe may be exposed when the pump door 102 is downturned. A display screen is mounted on the front side of the pump door 102, and when the pump door 102 is in the initial position, the user can see the display screen but cannot see the syringe. With the pump door 102 flipped down, the syringe can be seen, but the information on the screen cannot be seen. This prevents the drug label and the information on the screen itself from being viewed at the same time, which reduces clinical efficiency and is not conducive to observation and recording by clinical healthcare professionals.

The invention provides a screen control method for a syringe pump, apparatus, device, and storage medium, which are intended to solve the technical problems of the prior art as described above.

The technical solutions of the invention and how the technical solutions of the invention solve the technical problems as described above are described in detail in the following specific embodiments. These following specific embodiments may be combined with each other, and the same or similar concepts or processes may not be repeated in certain embodiments. Embodiments of the invention will be described below in conjunction with the accompanying drawings.

FIG. 2 is a flowchart of a screen control method for a syringe pump according to an embodiment of the invention. The screen control method is applied to a syringe pump, and specifically to a pump gate in a syringe pump, performed by a screen control device of the syringe pump. The syringe pump includes a pump door and a pump body, the pump door being mounted with a display screen, the pump body being configured to receive a syringe, and the pump door being slidably connected to the pump body. As shown in FIG. 2, the method includes the following steps:

S201, a screen control instruction corresponding to a sliding operation is determined according to the sliding operation performed by a user on the pump door. The sliding operation indicates sliding the pump door.

For example, the syringe pump may be used for administering a drug to a patient, and the user may prepare the syringe pump by mounting a syringe in the syringe pump, and the user may be a healthcare professional or the like. The syringe pump may include a pump body and a pump door, and a syringe may be placed on the pump body, for example, the pump body may be provided with a recess in which the syringe is placed. The pump door may be slidably connected to the pump body, with the pump body not sliding and the pump door sliding. The pump door is provided with a display screen, and the display screen is located on the outside of the pump door for viewing by a user. The pump door can be slid up and down relative to the pump body, or slid diagonally upward or diagonally downward. In this embodiment, the sliding direction of the pump door is not specifically limited. For example, a slide rail is provided on the left and right sides of the pump body, and a pulley is provided on the left and right sides of the pump door corresponding to the position of the slide rail, so that the pump door can slide up and down along the slide rail through the pulley.

FIG. 3 shows a schematic diagram of a syringe pump with a sliding pump door. In FIG. 3, the pump door 302 is located above the pump body 301, and slide rails 303 are provided on the left and right sides of the pump body 301, and the pump door 302 can slide up and down along the slide rails 303. The syringe is located in the pump body 301, and when the pump door 302 is located in an initial position, the syringe is completely obscured; when the pump door 302 is slid up and down, the user can see the syringe in the pump body 301.

The user can perform a sliding operation on the pump door. For example, the user can push the pump door to slide. When the user carries out a sliding action on the pump door, the syringe pump may determine sliding information for the pump door in response to the sliding operation. The sliding information may include a sliding direction, a sliding distance, and the like. Different sliding operations may correspond to different screen control instructions, for example, different sliding information corresponds to different screen control instructions. The screen control instructions refer to instructions that control the display state and display information of the screen, for example, the screen control instructions may include a power-on instruction, a power-off instruction, an information display instruction, and the like. The display state may include a power-on state, a power-off state, a lock screen state, and an unlock state, etc., and the display information may include information on a screen menu, user information, information relating to injection progress, and drug information, etc. Different sliding operations corresponding to different screen control instructions may mean that different sliding directions correspond to different screen control instructions, and/or that different sliding distances correspond to different screen control instructions. For example, if the user pushes the pump door to slide upward, the screen control instruction is determined to be a power-on instruction. For example, if the user pushes the pump door to slide upward by more than 10 cm, the corresponding screen control instruction is determined to be the power-on instruction.

S202, the display screen is controlled according to the screen control instruction.

For example, after determining the screen control instruction, the display screen is automatically controlled according to the screen control instruction. For example, if the screen control instruction is a power-on instruction, the display screen automatically carries out power-on. If the screen control instruction is an injection progress display instruction, the display screen carries out the display of the injection progress. That is, the user does not need to manually click on the display screen to trigger the operation, but sends the screen control command to the display screen while pushing the pump door. When the display screen performs the display, the determined sliding operation may also be displayed on the screen for the user to determine whether the sliding operation determined by the syringe pump is consistent with the sliding operation actually performed by the user.

In this embodiment, the step of controlling the display screen according to the screen control instruction includes: determining, according to the screen control instruction and a predetermined correlation relationship between the instruction and the display information, display information corresponding to the screen control instruction; and displaying the display information corresponding to the screen control instruction on the display screen.

Specifically, the display screen on the syringe pump may display different display information according to the screen control instructions, and the display information is the information to be displayed on the display screen. Correlation relationships between different screen control instructions and the display information are preset, and after the screen control instructions are determined, the display information corresponding to the screen control instructions is determined according to the preset correlation relationships. The determined display information is displayed on the display screen for the user to view. For example, if the screen control instruction is a drug information display instruction, the corresponding display information may include the name of the drug, the date of manufacture, the manufacturer, and the like.

The display information may also be picture information, for example, the display information may be a preset lock screen picture, and if the screen control instruction is a lock screen instruction, the preset lock screen picture is displayed on the screen. The screen control instruction may also correspond to, without displaying any information, for example, if the screen control instruction is a shutdown instruction, the display screen automatically carries out shutdown without displaying any information.

The beneficial effect of such a setting is that, by means of the screen control instruction, the display information of the display screen is determined, and the display information is automatically displayed, which does not require the user to carry out an additional operation, reduces the user's operation process, and improves the efficiency of the screen control of the syringe pump.

Embodiments of the invention provide a screen control method for a syringe pump, in which the syringe pump includes a pump door and a pump body, and a display screen is mounted on the pump door to display information such as the injection process and the injected drug. The pump body is configured to receive a syringe, the pump door is slidably connected to the pump body, and the syringe in the pump body can be exposed after the pump door is slid. The user can perform a sliding operation on the pump door, and different sliding operations can correspond to different screen control instructions, according to which the screen control instructions can realize automatic control of the display screen, thereby solving the problem of low control efficiency caused by the existing technology in which the user needs to manually make clicks and other operations on the screen for control. Moreover, by sliding the pump door to control the screen, it can satisfy the user's need to observe the information on the screen and the information on the syringe at the same time, thereby improving the control efficiency of the screen and enhancing the user's experience.

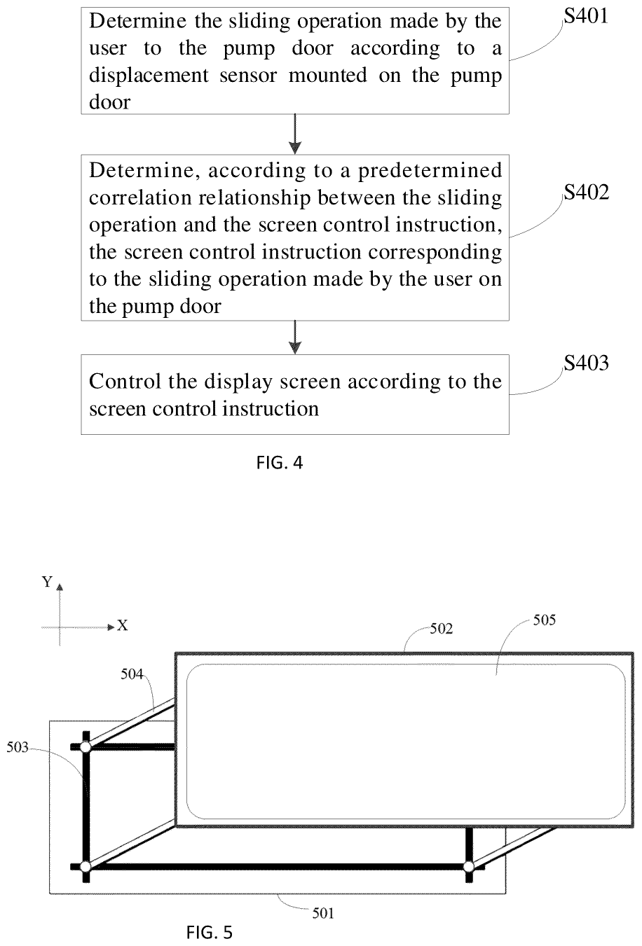

FIG. 4 shows a flowchart of a screen control method for a syringe pump according to an embodiment of the invention, which is an optional embodiment according to the above embodiment.

In this embodiment, the step of determining, according to a sliding operation performed by the user on the pump door, a screen control instruction corresponding to the sliding operation may be refined as: determining a sliding action carried out by the user on the pump door according to a displacement sensor mounted on the pump door; and determining a screen control instruction corresponding to the sliding operation performed by the user on the pump door according to a predetermined correlation relationship between the sliding operation and the screen control instruction.

As shown in FIG. 4, the method includes the following steps.

S401, the sliding operation performed by the user to the pump door is determined according to the displacement sensor mounted on the pump door.

For example, the pump door may be arranged with a displacement sensor, and the displacement sensor may sense the movement of the pump door and determine sliding information such as the sliding direction and the sliding distance of the pump door. The displacement sensor can determine the sliding information in real time to determine the sliding operation done by the user on the pump door. For example, if the sliding information is that the sliding direction is upward and the sliding distance is 12 cm, the sliding operation is determined as the pump door sliding upward by 12 cm.

In this embodiment, determining the sliding operation performed by the user on the pump door according to the displacement sensor mounted on the pump door includes: determining a current sliding direction of the pump door according to the displacement sensor mounted on the pump door; and determining, according to a predetermined correlation relationship between the sliding direction and the sliding operation, the sliding operation corresponding to the current sliding direction as the sliding operation performed by the user on the pump door.

Specifically, different correlation relationships between the sliding direction and the sliding operation may be preset, i.e., it may be possible to determine only the sliding direction without determining the sliding distance of the pump door. A sliding distance threshold for the pump door may be preset, and as long as the user pushes the pump door to slide, the pump door will slide the preset sliding distance threshold. For example, the sliding distance threshold for the pump door to slide upward may be 15 cm, and if the user applies an upward sliding force to the pump door, the pump door automatically slides 15 cm and stops sliding. After the pump door slides 15 cm, the pump door can no longer slide upward.

When the pump door slides, the displacement sensor can determine the sliding direction of the pump door in real time as the current sliding direction. According to a preset correlation relationship between the sliding direction and the sliding operation, a sliding operation corresponding to the current sliding direction is determined, i.e., a sliding operation performed by the user on the pump door is obtained. For example, if the displacement sensor determines that the current sliding direction is upward, the corresponding sliding operation can be determined to be the user pushing the pump door upward for sliding. If a sliding distance threshold is preset, it can be determined that the sliding operation is the user pushing the pump door upward to slide the distance of the preset sliding distance threshold.

The beneficial effect of such a setting is that the sliding operation of the pump door can be quickly determined according to the displacement sensor, improving the efficiency of determining the screen control instructions, and thus improving the control efficiency of the screen.

S402: According to the predetermined correlation relationship between the sliding operation and the screen control instruction, the screen control instruction corresponding to the sliding operation performed by the user to the pump door is determined.

For example, after determining the sliding operation, the screen control instruction corresponding to the sliding operation is determined. The correlation relationship between different sliding operations and the screen control instructions may be preset, and according to the correlation relationship, the corresponding screen control instruction is quickly determined. For example, if the pump door is slid upward, the corresponding screen control instruction could be a power-on instruction; if the pump door is slid rightward, the corresponding screen control instruction could be an unlock instruction.

In this embodiment, the step of determining the screen control instruction corresponding to the sliding operation performed by the user to the pump door according to the preset correlation relationship between the sliding operation and the screen control instruction includes: determining a sliding direction and a sliding distance in the sliding operation performed by the user to the pump door; and determining the screen control instruction corresponding to the sliding direction and the sliding distance according to the preset correlation relationship between the sliding operation and the screen control instruction. The screen control instruction includes a power-on instruction, a power-off instruction, a lock screen instruction, an unlock instruction, a menu information display instruction, and an injection instruction.

For example, the sliding operation may include information such as the sliding direction and the sliding distance, and the sliding direction, with the sliding distance being determined in real time by a displacement sensor. The predetermined correlation relationship between the sliding operation and the screen control instruction may be a correlation relationship between the sliding direction and the screen control instruction, and/or a correlation relationship between the sliding distance and the screen control instruction. It is also possible to combine the sliding direction and the sliding distance to preset a combined correlation relationship between the sliding direction and the sliding distance and the screen control instruction. For example, if the sliding direction is upward and the sliding distance is 10 cm, the corresponding screen control instruction could be a power-on instruction; if the sliding direction is upward and the sliding distance is 20 cm, the corresponding screen control instruction could be a menu information display instruction.

After obtaining the sliding direction and the sliding distance, the screen control instruction corresponding to the sliding direction and the sliding distance is determined according to the preset correlation relationship. The screen control instruction may include a power-on instruction, a power-off instruction, a lock screen instruction, an unlock instruction, a menu information display instruction, an injection instruction, and the like. The menu information display instruction may refer to displaying the menu information of the display screen, which makes it easier for the user to carry out subsequent operations. The injection instruction may refer to the display screen showing that the syringe is starting to carry out the injection, and the injection pump is controlling the syringe to start carrying out the injection. That is, if the screen control command is the injection command, the syringe automatically injects the drug into the patient, and the display screen shows the message “injection started” for the user to view.

The beneficial effect of this setup is that the user can issue a variety of screen control commands by simply sliding the pump door, reducing the complexity of the user's operation process, improving the efficiency of screen control, and enhancing the user's experience.

S403, according to the screen control instructions, the display screen is controlled.

For example, this step can be referred to as in step S202 above, and will not be repeated.

Embodiments of the invention provide a screen control method for a syringe pump, in which the syringe pump includes a pump door and a pump body, and a display screen is mounted on the pump door to display information such as the injection process and the injected drug. The pump body is configured to receive a syringe, the pump door is slidably connected to the pump body, and the syringe in the pump body can be exposed after the pump door is slid. The user can slide the pump door, and different sliding operations can correspond to different screen control instructions, according to which the screen control instructions can realize automatic control of the display screen, thereby solving the problem of low control efficiency caused by the existing technology in which the user needs to manually make clicks and other operations on the screen for control. Moreover, by sliding the pump door to control the screen, it can satisfy the user's need to observe the information on the screen and the information on the syringe at the same time, thereby improving the control efficiency of the screen and enhancing the user's experience.

FIG. 5 is a schematic diagram of a syringe pump with a sliding pump door according to an embodiment of the invention, which is an optional embodiment . As shown in FIG. 5, the syringe pump includes a support rod 504 connected between the pump door 502 and a slide rail 503 on the pump body 501, which is configured to support the pump door 502, so that the pump door 502 can be pulled out in a direction perpendicular to the pump body 501. The pump door 502 is configured to slide in parallel along the slide rail 503 on the pump body 501 by the support rod 504.

For example, the support rod 504 may be a material with a certain degree of hardness, such as metal, plastic, etc., and may connect the pump door 502 and the pump body 501 so that the pump door 502 is in a relatively parallel position with the pump body 501. The support rod 504 can support the pump door 502 to be displaced in a limited range of upward, downward, leftward, rightward and other directions in the parallel plane, while ensuring a stable connection between the pump door 502 and the pump body 501 of the syringe pump. The support rod 504 may be a telescopic structure, for example, the support rod 504 may be contracted into the pump body 501, and when the display screen 505 on the pump door 502 needs to be pulled out the support rod 504 may be pulled out perpendicular to the XY plane, i.e., perpendicular to the pump body 501. The support rod 504 may also be telescoped between the pump body 501 and the pump door 502, for example, the support rod 504 can be shortened to a predetermined minimum distance such that the pump door 502 obscures the syringe in the pump body 501. The support rod 504 may also be a connecting rod structure, for example, the support rod 504 may be folded in the slide rail 503 of the pump body 501, and when the display screen 505 needs to be pulled out, the support rod 504 may be straightened from the folded state, and the straightened support rod 504 is perpendicular to the pump body 501. The support rod 504 may support the pump door 502 to slide along the slide rail on the pump body 501, in parallel. Parallel sliding means that, when the pump door 502 is sliding, the plane in which the pump door 502 is located is parallel to the plane where the pump body 501 is located.

In this embodiment, the pump door is preset with an initial position, and the pump door obscures the syringe in the pump body at the initial position; the pump door is configured to display the syringe in the pump body when the user slides the pump door.

For example, an initial position of the pump door 502 may be preset, with the initial position being a position at which the pump door 502 completely obscures the syringe in the pump body 501; for example, the preset position may be a position at which the pump door 502 is located when the support bar 504 is not pulled out. When a sliding operation is performed on the pump door 502, the syringe in the pump body 501 will be visible, i.e., the user can see the syringe in the pump body 501 after sliding the pump door 502. For example, if the syringe pump does not include the support rod 504, the sliding operation performed by the user may be to push the pump door 502 to slide. The pump door 502 is first located in an initial position, and the user pushes the pump door 502 upward, and the syringe pump reveals the syringe in the pump body 501. If the syringe pump includes a support rod 504, the sliding operation may include a pull-out operation of pulling the support rod 504 out and an operation of pushing the pump door 502 to slide after pulling it out. The pump door 502 is initially positioned just to cover the syringe, and there may be no gap between the pump door 502 and the pump body 501 in the initial position. When the user pulls out the pump door 502 through the support rod 504, the pump door 502 leaves the initial position, and the user can see the syringe in the pump body 501 and can slide the pump door 502.

The beneficial effect of such a setting is that the initial position of the pump door is preset, so that the pump door can cover the syringe in the initial position, realizing the protection and concealment of the syringe. When the pump door slides, the syringe is revealed so that the user can see the syringe while viewing the display screen, satisfying the user's need to simultaneously observe the information on the screen and the information on the syringe, and enhancing the user's experience.

In this embodiment, the pump door and the pump body are connected by a slide rail and a slider; the pump door is configured to slide parallel to the pump body, along the slide rail, by means of the slider.

For example, the pump body 501 may be provided with a slide rail 503, and if the syringe pump does not include a support rod 504, the pump door 502 can be fixed with a slider, and the slider can be slid on the slide rail 503, so that the pump door 502 can be slid in parallel relative to the pump body 501. When the pump door 502 is in an initial position, the slider on the pump door 502 is on the slide rail 503, so that there is no excessive gap between the pump door 502 and the pump body 501, and the pump door 502 can cover the syringe in the pump body 501.

If a support rod 504 is included on the syringe pump, the slide may be disposed at one end of the support rod 504, and the other end of the support rod 504 may be fixed to the pump door 502. When the support rod 504 is pulled out in a direction perpendicular to the XY plane, the end of the support rod 504 will be fixed at the intersection point with the slide 503, and then the pump door 502 together with the display screen 504 can be slid autonomously along the fixed plane of the pump body 501 to adjust the position. The end of the support rod 504 may refer to the end where the slider is located, i.e., the end of the support rod 504 passes through the slider and slides in the slide rail 503.

The beneficial effect of such a setting is that the sliding of the pump door is realized through the slide rail and the slider, so that the user can see the display screen and the syringe at the same time, and the efficiency of the use of the syringe pump is improved.

In this embodiment, before determining a screen control instruction corresponding to the sliding operation according to the sliding operation performed by the user on the pump door, it further includes: activating a displacement sensor mounted on the pump door in response to a pull-out operation performed by the user to the pump door, so as to determine the sliding operation carried out by the user on the pump door according to the displacement sensor mounted on the pump door.

For example, the user may make a pull-out operation on the support rod 504 to pull the support rod 504 out to straighten. The syringe pump may be provided with a preset sensor for sensing a state of the support rod 504, and the state of the support rod 504 may include a pull-out state and a retracted state, where the pull-out state means that the support rod 504 pulls the pump door 502 out from an initial position, and the retracted state means that the support rod 504 retracts the pump door 502 to an initial position. If the syringe pump responds to a user pulling out the pump door 502 via a preset sensor, i.e., it is determined that the support rod 504 changes from the retracted state to the pulled-out state, and the displacement sensor on the pump door 502 is activated. That is, the displacement sensor does not operate when the support rod 504 is in the retracted state, and the displacement sensor is activated to operate when the support rod 504 is in the pulled-out state.

After the displacement sensor is activated, the displacement sensor can determine the sliding information of the pump door 502 in real time, so as to obtain the sliding operation performed by the user to the pump door 502.

The beneficial effect of such a setting is that activating the displacement sensor after pulling out the support rod reduces the waste of electricity of the syringe pump and realizes the efficient use of the syringe pump.

The syringe pump in the embodiments of the invention enables users such as healthcare personnel to conveniently operate the syringe pump, while viewing information such as the screen information of the syringe pump and the drug labels of the syringes in the pump body, and the user can carry out use operations such as powering on, powering off, locking the screen, and unlocking the screen by sliding the screen to improve the work efficiency of the healthcare personnel, and to facilitate the observation and recording by the healthcare personnel.

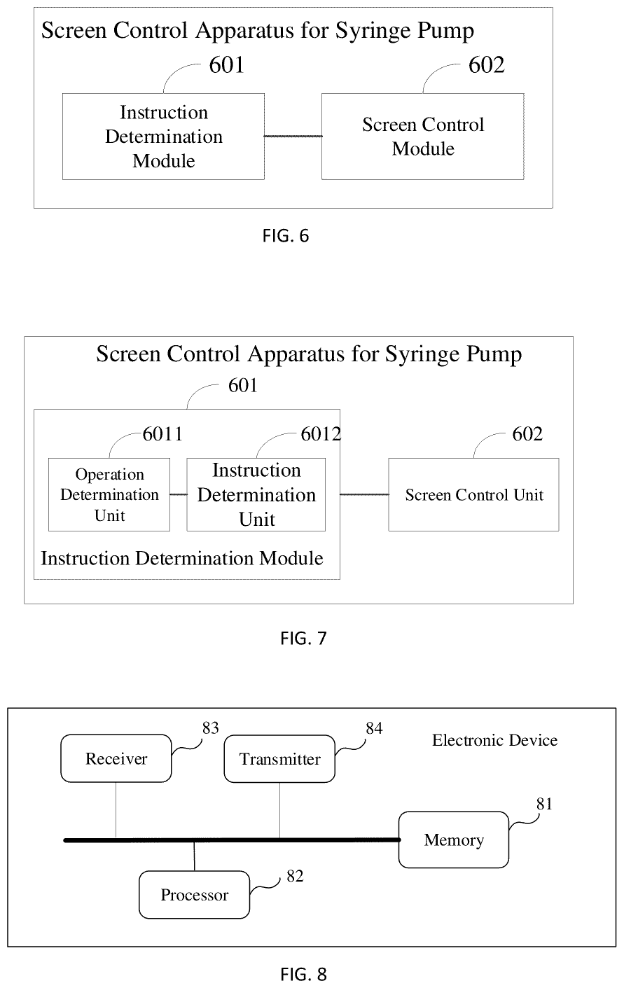

FIG. 6 shows a block diagram of a screen control apparatus for a syringe pump according to an embodiment of the invention, which is applied to a syringe pump, in particular, to a pump door in a syringe pump. The syringe pump includes a pump door and a pump body, in which the pump door is fitted with a display screen, the pump body is configured to place a syringe, and the pump door is slidably connected to the pump body. For ease of illustration, only those portions relevant to embodiments of the present invention are shown. Refer to FIG. 6, which shows the apparatus as including: an instruction determination module 601 and a screen control module 602.

The command determination module 601 is configured to determine a screen control command corresponding to the sliding operation according to a sliding operation performed by a user on the pump door. The sliding operation indicates sliding of the pump door;

The screen control module 602 is configured to control the display screen according to the screen control instruction.

FIG. 7 shows a block diagram of a screen control device for a syringe pump according to an embodiment of the invention. On the basis of the embodiment shown in FIG. 6, as shown in FIG. 7, the instruction determination module 601 includes an operation determination unit 6011 and an instruction determination unit 6012.

The operation determination unit 6011 is configured to determine a sliding operation performed by a user on the pump door according to a displacement sensor mounted on the pump door.

The instruction determination unit 6012 is configured to determine a screen control instruction corresponding to a sliding operation performed by a user to the pump door according to a predetermined correlation relationship between the sliding operation and the screen control instruction.

In one embodiment, the operation determination unit 6011, is configured to:
determine a current sliding direction of the pump door according to a displacement sensor mounted on the pump door; and
determine a sliding operation corresponding to the current sliding direction as a sliding operation performed by a user on the pump door, according to a predetermined correlation relationship between the sliding direction and the sliding operation.

In one embodiment, the command determination unit 6012, is configured to:
determine a sliding direction and a sliding distance in a sliding operation performed by the user to the pump door; and
determine a screen control instruction corresponding to the sliding direction and sliding distance according to a preset correlation relationship between the sliding operation and a screen control instruction. The screen control instruction includes a power-on instruction, a power-off instruction, a lock screen instruction, an unlock instruction, a menu information display instruction, or an injection instruction.

In one embodiment, the pump door is preset with an initial position and the pump door shields a syringe in the pump body when in the initial position.

The pump door is configured to reveal a syringe in the pump body when the user makes a sliding operation on the pump door.

In one embodiment, the pump door is connected to the pump body by means of a slide and a slider;

The pump door is configured to slide parallel to the pump body along the slide rail by the slider.

In one embodiment, the syringe pump includes a support rod, the support rod being connected between the pump door and a slide rail on the pump body.

The support rod is configured to support the pump door by pulling the pump door out in a direction perpendicular to the pump body;

The pump door is configured to slide parallel along a slide rail on the pump body by the support rod.

In one embodiment, the device further includes:
a pump door pull-out module configured to activate a displacement sensor mounted on the pump door in response to a pull-out operation performed by a user on the pump door before determining a screen control command corresponding to the sliding operation according to a sliding operation performed by a user on the pump door to determine a sliding operation performed by a user on the pump door according to a displacement sensor mounted on the pump door.

In one embodiment, a screen control module 602, includes:
an information determination unit configured to determine display information corresponding to the screen control instruction according to a preset correlation relationship between the instruction and the display information according to the screen control instruction;
an information display unit configured to display the display information corresponding to the screen control instruction on the display screen.

Embodiments of the invention provide a syringe pump, and the syringe pump may be used to perform the screen control method for the syringe pump described in any embodiment of the invention.

FIG. 8 shows a block diagram of an electronic device provided by embodiments of the invention. As shown in FIG. 8, the electronic device includes: a memory 81, a processor 82. The memory 81 is configured to store executable instructions of the processor 82.

The processor 82 is configured to execute a method as provided in the above embodiment.

The electronic device further includes a receiver 83 and a transmitter 84. The receiver 83 is configured to receive instructions and data sent by other devices, and the transmitter 84 is configured to send instructions and data to an external device.

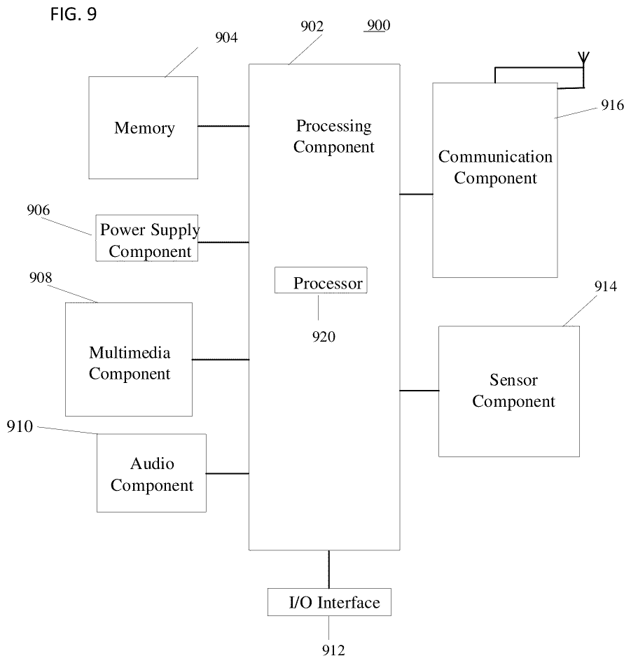

FIG. 9 is a block diagram of an electronic device illustrated according to an example embodiment, which may be a cell phone, a computer, a digital broadcasting terminal, a message transceiver device, a game console, a tablet device, a medical device, a fitness device, a personal digital assistant, a vehicle, and the like.

The device 900 may include one or more of the following components: a processing component 902, a memory 904, a power supply component 906, a multimedia component 908, an audio component 910, an input/output (I/O) interface 912, a sensor component 914, and a communication component 916.

The processing component 902 generally controls the overall operation of the device 900, such as operations associated with displays, telephone calls, data communications, camera operations, and recording operations. The processing component 902 may include one or more processors 920 to execute instructions to accomplish all or some of the steps of the method described above. In addition, the processing component 902 may include one or more modules to facilitate interaction between the processing component 902 and other components. For example, the processing component 902 may include a multimedia module to facilitate interaction between the multimedia component 908 and the processing component 902.

The memory 904 is configured to store various types of data to support operations at the device 900. Examples of such data include instructions for any application or method operated on the device 900, contact data, phone book data, messages, pictures, videos, etc. The memory 904 may be implemented by any type of volatile or non-volatile storage device or a combination thereof, such as static random access memory (SRAM), electrically erasable programmable read-only memory (EEPROM), erasable programmable read-only memory (EPROM), programmable read-only memory (PROM), read-only memory (ROM), magnetic memory, flash memory, a disk or CD-ROM.

The power supply assembly 906 provides power to the various components of the device 900. The power supply component 906 may include a power management system, one or more power supplies, and other components associated with generating, managing, and distributing power for the device 900.

The multimedia component 908 includes a screen providing an output interface between the device 900 and a user. In some embodiments, the screen may include a liquid crystal display (LCD) and a touch panel (TP). If the screen includes a touch panel, the screen may be implemented as a touch screen to receive input signals from the user. The touch panel includes one or more touch sensors to sense touches, swipes and gestures on the touch panel. The touch sensors may not only sense the boundaries of a touch or swipe action, but also detect the duration and pressure associated with the touch or swipe operation. In some embodiments, the multimedia component 908 includes a front camera and/or a rear camera. The front camera and/or the rear camera may receive external multimedia data when the device 900 is in an operating mode, such as a shooting mode or a video mode. Each front camera and rear camera may be a fixed optical lens system or have a focal length and optical zoom capability.

The audio component 910 is configured to output and/or input audio signals. For example, the audio component 910 includes a microphone (MIC) that is configured to receive external audio signals when the device 900 is in an operational mode, such as a call mode, a recording mode, and a voice recognition mode. The received audio signals may be further stored in memory 904 or sent via communication component 916. In some embodiments, the audio component 910 further includes a speaker for outputting the audio signal.

The I/O interface 912 provides an interface between the processing component 902 and a peripheral interface module, the peripheral interface module may be a keypad, click wheel, buttons, etc. These buttons may include, but are not limited to: a home button, a volume button, a start button, and a lock button.

The sensor assembly 914 includes one or more sensors for providing an assessment of various aspects of the status of the device 900. For example, the sensor assembly 914 may detect an open/closed state of the device 900, the relative positioning of components, such as the components being the display and keypad of the device 900, and the sensor assembly 914 may also detect a change in the position of the device 900 or a component of the device 900, the presence or absence of user contact with the device 900, the orientation or acceleration/deceleration of the device 900, and a device 900 a change in temperature of the device 900. The sensor assembly 914 may include a proximity sensor configured to detect the presence of a nearby object in the absence of any physical contact. The sensor assembly 914 may also include a light sensor, such as a CMOS or CCD image sensor, for use in imaging applications. In some embodiments, the sensor assembly 914 may also include an accelerometer sensor, a gyroscope sensor, a magnetic sensor, a pressure sensor, or a temperature sensor.

The communication component 916 is configured to facilitate communication between the device 900 and other devices by wired or wireless means. The device 900 may access a wireless network according to a communication standard, such as WiFi, 2G or 3G, or a combination thereof. In an exemplary embodiment, the communication component 916 receives broadcast signals or broadcast-related information from an external broadcast management system via a broadcast channel. In an exemplary embodiment, the communication component 916 further includes a near field communication (NFC) module to facilitate short range communication. For example, in the NFC module may be implemented according to Radio Frequency Identification (RFID) technology, Infrared Data Association (IrDA) technology, Ultra Wide Band (UWB) technology, Bluetooth (BT) technology and other technologies.

In exemplary embodiments, the device 900 may be implemented by one or more application-specific integrated circuits (ASICs), digital signal processors (DSPs), digital signal processing devices (DSPDs), programmable logic devices (PLDs), field-programmable gate arrays (FPGAs), controllers, microcontrollers, microprocessors, or other electronic components for performing the methods described above.

In exemplary embodiments, there is also provided a non-transitory computer-readable storage medium including instructions, such as a memory 904 including instructions, the instructions being executable by a processor 920 of a device 900 to accomplish the method. For example, the non-transitory computer readable storage medium may be a ROM, random access memory (RAM), CD-ROM, magnetic tape, floppy disk, and optical data storage device, among others.

A non-transitory computer-readable storage medium that, when instructions in the storage medium are executed by a processor of a terminal device, enables the terminal device to perform the screen control method for a syringe pump of the terminal device.

The invention also discloses a computer program product including a computer program that when executed by a processor implements a method as described in this embodiment.

Various embodiments of the systems and techniques described above in the invention can be found in digital electronic circuit systems, integrated circuit systems, field programmable gate arrays (FPGAs), application-specific integrated circuits (ASICs), application-specific standardized products (ASSPs), systems of systems-on-a-chip (SOCs), load-programmable logic devices (CPLDs), computer hardware, firmware, software, and/or combinations thereof implementations. These various embodiments may include:

implementation in one or more computer programs that may be executed and/or interpreted on a programmable system including at least one programmable processor, which may be a dedicated or general-purpose programmable processor, that may receive data and instructions from the storage system, the at least one input device, and the at least one output device, and transmit the data and instructions to the storage system, the at least one input device, and the at least one output device.

Program code for implementing the methods of the invention may be written in any combination of one or more programming languages. Such program code may be provided to a processor or controller of a general-purpose computer, a special-purpose computer, or other programmable data processing device such that the program code when executed by the processor or controller causes the functions/operations set forth in the flowchart and/or the block diagram to be implemented. The program code may be executed entirely on the machine, partially on the machine, partially on the machine as a stand-alone software package and partially on a remote machine or entirely on a remote machine or electronic device.

In the context of the invention, a machine-readable medium may be a tangible medium that may contain or store a program for use by or in conjunction with an instruction execution system, device, or apparatus. The machine-readable medium may be a machine-readable signal medium or a machine-readable storage medium. The machine-readable medium may include, but is not limited to, an electronic, magnetic, optical, electromagnetic, infrared, or semiconductor system, device, or apparatus, or any suitable combination of the foregoing. More specific examples of machine-readable storage media would include electrical connections based on one or more wires, portable computer disks, hard disks, random access memory (RAM), read-only memories (ROM), erasable programmable read-only memories (EPROMs or flash memories), optical fibers, convenient compact disk read-only memories (CD-ROMs), optical storage devices, magnetic storage devices, or any suitable combination of the foregoing.

To provide interaction with a user, the systems and techniques described herein may be implemented on a computer having: a display device (e.g., a CRT (cathode ray tube) or LCD (liquid crystal display) monitor) for displaying information to the user; and a keyboard and pointing device (e.g., a mouse or trackball) by which the user can provide input to the computer. Other types of devices may also be used to provide input to the computer. Other types of devices may also be used to provide interaction with the user; for example, the feedback provided to the user may be any form of sensory feedback (e.g., visual feedback, auditory feedback, or haptic feedback); and the input from the user may be received in any form (including acoustic input, voice input, or haptic input).

The systems and techniques described herein can be implemented in a computing system that includes back-end components (e.g., as a data electronic device), or a computing system that includes middleware components (e.g., an application electronic device), or a computing system that includes front-end components (e.g., a user's computer that has a graphical user interface (GUI) or a web browser through which a user can communicate with a user's computer, a user's computer, a user's computer, a user's computer, and a user's computer. embodiments of the systems and techniques described herein), or in a computing system that includes any combination of such back-end components, middleware components, or front-end components. The components of the system may be interconnected by digital data communications (e.g., a communications network) in any form or medium. Examples of communication networks include: local area networks (LANs), wide area networks (WANs), and the Internet.

A computer system may include a client and an electronic device. Clients and electronic devices are generally remote from each other and typically interact over a communications network. The client-electronic device relationship is generated by a computer program that runs on a corresponding computer and has a client-electronic device relationship with each other. An electronic device may be a cloud electronic device, also known as a cloud computing electronic device or a cloud host, which is a host product in a cloud computing service system to address the shortcomings of traditional physical hosts and Virtual Private Servers (VPS), which are difficult to manage and weak in terms of business scalability. Electronic devices can also be electronic devices for distributed systems, or electronic devices that incorporate blockchain. It should be understood that steps can be reordered, added, or removed using various forms of processes shown above. For example, the steps documented in the invention may be performed in parallel or sequentially or in a different order, as long as they achieve the desired results of the technical solutions disclosed in the invention, and are not limited herein.

Other embodiments of the invention will readily come to mind to those skilled in the art upon consideration of the specification and practice of the invention disclosed herein. The invention is intended to cover any variations, uses, or adaptations of the invention that follow the general principles of the invention and include common knowledge or customary technical means in the art not disclosed herein. The specification and embodiments are to be regarded as exemplary only, and the true scope and spirit of the invention is indicated by the following claims.

It is to be understood that the invention is not limited to the precise structure which has been described above and illustrated in the accompanying drawings, and that various modifications and changes may be made without departing from its scope. The scope of the invention is limited only by the appended claims.

1. A screen control method for a syringe pump that is configured to receive a syringe and has a pump door, and a pump body, the method comprising:
determining, according to a sliding operation performed by a user on the pump door, a screen control instruction corresponding to the sliding operation; said sliding indicate sliding of the pump door relative to the pump body; and
controlling a display screen mounted in the pump door according to the screen control instruction.

2. The method of claim 1, wherein the step of determining the screen control instruction comprises:
determining the sliding operation performed by the user on the pump door according to a displacement sensor mounted on the pump door; and
determining, according to a predetermined correlation relationship between the sliding operation and the screen control instruction, the screen control instruction corresponding to the sliding operation performed by the user on the pump door.

3. The method of claim 2, wherein the step of determining the sliding operation comprises:
determining a current sliding direction of the pump door according to the displacement sensor mounted on the pump door; and
determining, according to a predetermined correlation relationship between the sliding direction and the sliding operation, the sliding operation corresponding to the current sliding direction as the sliding operation performed by a user on the pump door.

4. The method of claim 2, wherein the step of determining the screen control instruction comprises:
determining a sliding direction and a sliding distance of the sliding operation performed by the user to the pump door; and
determining a screen control instruction corresponding to the sliding direction and sliding distance according to the predetermined correlation relationship between the sliding operation and the screen control instruction;
said screen control instruction comprising at least one of a power-on instruction, a power-off instruction, a lock screen instruction, an unlock instruction, a menu information display instruction, and an injection instruction.

5. The method of claim 1, in which:
the pump door has an initial position in which the pump door shields a syringe in the pump body; and
the pump door is configured to make visible the syringe in the pump body when the user performs the sliding operation on the pump door.

6. The method of claim 1, wherein the pump door is connected to the pump body by means of a slide rail and a slider; and
the pump door is configured to slide parallel to the pump body, via the slider, along the slide rail.

7. The method of claim 1, wherein
the syringe pump comprises a support rod that is connected between the pump door and a slide rail on the pump body;
the support rod is configured to support the pump door, so that the pump door is capable of being pulled out in a direction perpendicular to the pump body; and
the pump door is configured to slide parallel along the slide rail on the pump body by the support rod.

8. The method of claim 7, wherein before the step of determining the screen control instruction comprises:
activating a displacement sensor mounted on the pump door in response to a pull-out operation performed by the user on the pump door, and determining the sliding operation performed by the user on the pump door according to the displacement sensor .

9. The method of claim 1, wherein the step of controlling the display screen according to the screen control instructions comprises:
determining, according to the screen control instruction and a predetermined correlation relationship between instruction and display information, display information corresponding to the screen control instruction; and
displaying the display information corresponding to the screen control instruction on the display screen.

10. A screen control apparatus for a syringe pump, said syringe pump having a pump body and a pump door, comprising:
an instruction determination module configured to determine, according to a sliding operation performed by a user on the pump door, a screen control instruction corresponding to the sliding operation; wherein the sliding operation is configured to indicate sliding of the pump door; and
a screen control module configured to control a display screen mounted in the pump door according to the screen control instruction.

11. An electronic device, comprising a processor, and a memory in communication with the processor; wherein
the memory is configured to store computer-executable instructions; and
the processor is configured to execute the computer-executable instructions stored in the memory, said computer-executable instructions being provided to cause the processor to
determine, according to a sliding operation performed by a user on a pump door of a syringe pump, a screen control instruction corresponding to the sliding operation; wherein the sliding operation indicates sliding of the pump door; and
control the display screen according to the screen control instruction.

12. The electronic device of claim 11, wherein the processor is further configured to determine, according to the sliding operation performed by the user on the pump door, the screen control instruction corresponding to the sliding operation, by:
determining the sliding operation performed by the user to the pump door according to a displacement sensor mounted on the pump door; and
determining, according to a predetermined correlation relationship between the sliding operation and the screen control instruction, the screen control instruction corresponding to the sliding operation performed by the user on the pump door.

13. The electronic device of claim 12, wherein the processor is further configured to determine the sliding operation by:
determining a current sliding direction of the pump door according to the displacement sensor mounted on the pump door; and
determining, according to a predetermined correlation relationship between the sliding direction and the sliding operation, the sliding operation corresponding to the current sliding direction as the sliding operation performed by the user on the pump door.

14. The electronic device of claim 12, wherein the processor is further configured to determine the screen control instruction by:
determining a sliding direction and a sliding distance of the sliding operation performed by the user to the pump door; and
determining a screen control instruction corresponding to the sliding direction and sliding distance according to the predetermined correlation relationship between the sliding operation and the screen control instruction; wherein the screen control instruction comprises at least one of a power-on instruction, a power-off instruction, a lock screen instruction, an unlock instruction, a menu information display instruction, and an injection instruction.

15. The electronic device of claim 11, wherein the processor is further configured to control the display screen by:
determining, according to the screen control instruction and a predetermined correlation relationship between instruction and display information, display information corresponding to the screen control instruction; and
displaying the display information corresponding to the screen control instruction on the display screen.
